(19) United States
(12) Patent Application Publication
(10) Pub. No.: US 2024/0156412 A1
(43) Pub. Date: May 16, 2024

(54) VITAL SIGN OBTAINING DEVICE, NON-TRANSITORY COMPUTER READABLE STORAGE MEDIUM, AND VITAL SIGN OBTAINING SYSTEM

(71) Applicant: Nihon Kohden Corporation, Tokyo (JP)

(72) Inventor: Wataru Matsuzawa, Tokorozawa-shi (JP)

(21) Appl. No.: 18/501,255

(22) Filed: Nov. 3, 2023

(30) Foreign Application Priority Data
Nov. 14, 2022 (JP) 2022-181760

(51) Int. Cl. A61B 5/00; A61B 5/08; G16H 50/30

(52) U.S. Cl. CPC A61B 5/7264; A61B 5/08; A61B 5/4809; A61B 5/7203; A61B 5/7282; G16H 50/30

(57) ABSTRACT

A vital sign obtaining device includes an interface configured to receive, from a first sensor, a first detection signal corresponding to a vital sign of a subject, the first sensor configured to obtain the vital sign, an inference model configured to output a probability that the first detection signal is classified into each of a plurality of classes, and a processor configured to output data corresponding to a vital sign associated with the first detection signal, the probability that the first detection signal is classified into one of the plurality of classes being equal to or greater than a threshold.

### CROSS-REFERENCE TO RELATED APPLICATIONS

This application is based on and claims priority under 35 USC 119 from Japanese Patent Application No. 2022-181760 filed on Nov. 14, 2022, the entire content of which is incorporated herein by reference.

### TECHNICAL FIELD

The present disclosure relates to a device configured to obtain a vital sign from a subject and a non-transitory computer readable storage medium storing a computer program configured to be executed by a processor mounted on the device. The present disclosure also relates to a vital sign obtaining system including the device and a management device configured to manage attribute information of the subject.

### BACKGROUND ART

Various vital signs are obtained in order to grasp the condition of the subject. A medical worker periodically visits the subject and obtains a predetermined vital sign through an appropriate device. This work is called a spot check. JP2021-129916A discloses a device that supports spot check work for obtaining a respiration rate, which is an example of vital signs.

It is required to reduce a burden on a medical worker related to spot check work.

### SUMMARY OF INVENTION

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to a first aspect of the present disclosure, there is provided a vital sign obtaining device including:
an interface configured to receive, from a first sensor, a first detection signal corresponding to a vital sign of a subject, the first sensor configured to obtain the vital sign;
an inference model configured to output a probability that the first detection signal is classified into each of a plurality of classes; and
a processor configured to output data corresponding to a vital sign associated with the first detection signal, the probability that the first detection signal is classified into one of the plurality of classes being equal to or greater than a threshold.

According to a second aspect of the present disclosure, there is provided a non-transitory computer readable storage medium storing a computer program. the computer program including instructions which, when executed by a processor mounted on a vital sign obtaining device, cause the vital sign obtaining device to:
receive, from a sensor, a detection signal corresponding to a vital sign of a subject, the sensor configured to obtain the vital sign;
input the detection signal to an inference model, the inference model being configured to output a probability that the detection signal is classified into each of a plurality of classes; and
output data corresponding to a vital sign associated with the detection signal, the probability that the detection signal is classified into one of the plurality of classes being equal to or greater than a threshold, the probability being output from the inference model.

According to a third aspect of the present disclosure, there is provided a vital sign obtaining system including:
a first sensor configured to output a first detection signal corresponding to a vital sign of a subject;
a management device configured to manage attribute information of the subject;
an inference model configured to output a probability that the first detection signal is classified into each of a plurality of classes; and
a processor configured to output, to the management device, data corresponding to a vital sign associated with the first detection signal, the probability that the first detection signal is classified into one of the plurality of classes being equal to or greater than a threshold,
in which the management device is configured to store the data in association with the attribute information.

### BRIEF DESCRIPTION OF DRAWINGS

Exemplary embodiment(s) of the present invention will be described in detail based on the following figures, wherein:

FIG. 1 illustrates a functional configuration of a vital sign obtaining system according to an exemplary embodiment of the present invention; and

FIG. 2 illustrates a functional configuration of a vital sign obtaining system according to an exemplary embodiment of the present invention.

### DESCRIPTION OF EMBODIMENTS

Embodiments will be described in detail with reference to the accompanying drawings.

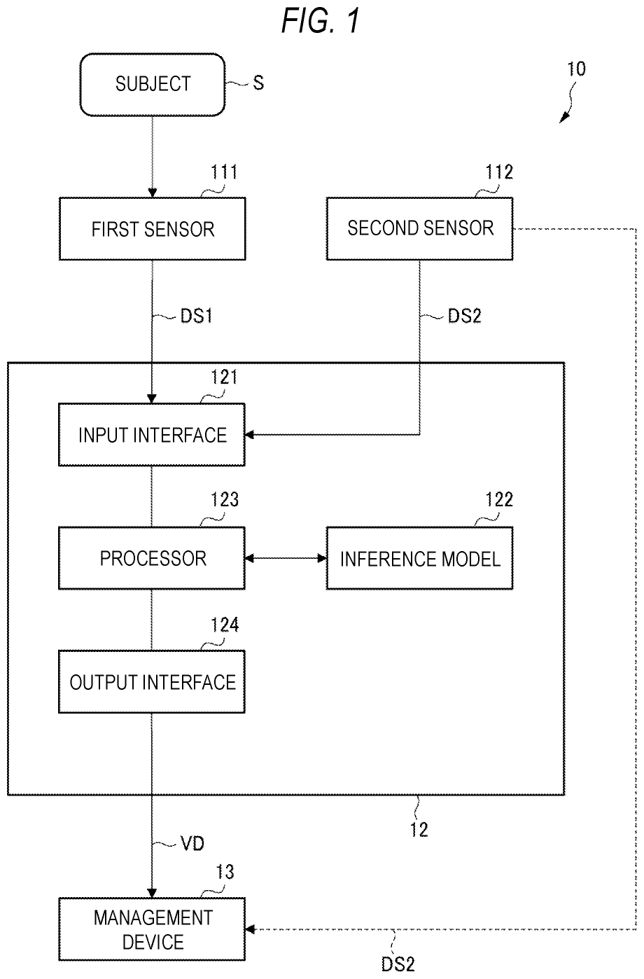

FIG. 1 illustrates a functional configuration of a vital sign obtaining system 10 according to an embodiment. The vital sign obtaining system 10 is a system for obtaining and managing a vital sign of a subject S.

The term “vital sign” used in the present specification means at least one numerical value of a pulse, respiration, a body temperature, a blood pressure, or a level of consciousness, or a change over time in the numerical value. The change over time in the numerical value may be visualized as a waveform or a graph.

The vital sign obtaining system 10 can include a first sensor 111. The first sensor 111 is configured to output a first detection signal DS1 corresponding to the vital sign of the subject S. The first detection signal DS1 may be an analog signal or a digital signal depending on a specification of the first sensor 111.

The first sensor 111 may have a known configuration according to the obtained vital sign. As an example, the first sensor 111 may be an unrestrained sensor installed on a hospital bed. In another example, the first sensor 111 may be mounted on a wearable device worn on the body of the subject S or on a mobile device that can be carried by the subject S. In another example, the first sensor 111 may be mounted on a monitor device, a medical telemeter, a camera, a thermal camera, a smart speaker, or the like installed in a medical facility.

In a case where the first sensor 111 is the unrestrained sensor installed on the hospital bed, the respiration rate or the heart rate may be obtained as the vital sign. In a case where the first sensor 111 is mounted on the camera or the smart speaker, the level of consciousness may be obtained as the vital sign. In a case where the first sensor 111 is mounted on the thermal camera, the body temperature may be obtained as the vital sign. In a case where the first sensor 111 is mounted on the wearable device or the mobile device, the heart rate, the respiration rate, the blood pressure, and the body temperature may be obtained as the vital signs.

The vital sign obtaining system 10 can include a processing device 12. The processing device 12 can include an input interface 121, an inference model 122, a processor 123, and an output interface 124. The processing device 12 is an example of the vital sign obtaining device.

The input interface 121 is configured as a hardware interface configured to receive the first detection signal DS1. In a case where the first detection signal DS1 is the analog signal, the input interface 121 can include an appropriate conversion circuit including an A/D converter. This description is same or similarly applied to other signals and data that can be received by the input interface 121 to be described later.

The inference model 122 is an algorithm configured to output, as an inference result, a probability that the first detection signal DS1 is classified into each of a plurality of classes. Examples of the plurality of classes include “with artifact” and “without artifact”. The “with artifact” class corresponds to a state in which a non-negligible artifact is superimposed on the first detection signal DS1. The “without artifact” class corresponds to a state in which no artifact is superimposed on the first detection signal DS1 or the artifact superimposed on the first detection signal DS1 is negligible. As another example of the plurality of classes, whether the subject S is in a sleep state, whether Cheyne-Stokes respiration, obstructive sleep apnea, central sleep apnea, or the like occurs in the subject S, or the like are exemplified. The probability takes a value between 0 and 1. The value 0 corresponds to 0%. The value 1 corresponds to 100%. The inference result may be a score (for example, any value from 1 to 5) corresponding to the probability of inference.

The inference model 122 can be generated by performing machine learning using training data. The training data may be appropriately generated according to the type of the obtained vital sign and a plurality of set classes. Examples of algorithms used for machine learning include a neural network, a decision tree, a random forest, and a support vector machine.

The processor 123 is configured to input, to the inference model 122, the first detection signal DS1 received by the input interface 121, and configured to obtain data corresponding to the probability output from the inference model 122. The processor 123 is configured to determine whether the probability that the first detection signal DS1 is classified into a specific class is equal to or greater than a threshold. For example, it is determined whether the probability that the first detection signal DS1 is classified into the “without artifact” class is equal to or greater than the threshold.

The processor 123 is configured to output, from the output interface 124, vital sign data VD corresponding to the vital sign associated with the first detection signal DS1, in which the probability that the first detection signal DS1 is determined to be equal to or greater than the threshold. The vital sign data VD may be in the form of analog data or in the form of digital data, according to a specification of the device configured to receive the data.

The output interface 124 is configured as a hardware interface. In a case where the vital sign data VD is in the form of analog data, the output interface 124 can include an appropriate conversion circuit including a D/A converter. This description is same or similarly applied to other signals and data that can be output by the output interface 124 to be described later.

The vital sign obtaining system 10 can include a management device 13. The management device 13 is configured to manage attribute information of the subject S. Examples of the attribute information include the name, age, gender, and past medical history of the subject S. That is, the management device 13 may be a part of a management database system such as an electronic medical record.

The management device 13 may include a storage such as a semi-conductor memory, a hard disk device, or a magnetic tape device. The management device 13 is configured to store, in the storage, the vital sign data VD of the subject S output from the processing device 12 in association with the attribute information of the subject S.

In the spot check work, a medical worker generally visits a hospital bed of the subject S several times per day and obtains a predetermined vital sign. The obtained vital sign is recorded in a management database such as an electronic medical record through manual work or equipment called a spot checker. This work is strongly restricted at regular intervals, but it is not always possible for the subject to obtain a vital sign excellently at the time of visit, which is a cause of an increase in a burden on the medical worker.

According to the above configuration, data corresponding to the vital sign of the subject S obtained through the first sensor 111 can be input to the processing device 12 as needed. In addition, data corresponding to a vital sign with a high probability that corresponds to one of a plurality of preset classes can be selectively obtained and stored in association with the attribute information of the subject S. In other words, it is possible to automatically obtain a vital sign to be recorded in the management database such as the electronic medical record, even when the medical worker does not visit the hospital bed of the subject S. In addition, by selecting a vital sign corresponding to a desired condition by the intervention of the inference model 122, the vital sign data VD that is recorded in a state in which an influence of the state of the subject S and the obtaining environment is suppressed can be obtained. Accordingly, it is possible to reduce the burden on the medical worker related to the spot check work.

In particular, by setting the plurality of classes such that the artifact superimposed on the vital sign is associated therewith, for example, the vital sign data VD corresponding to the vital sign in which the superimposition of the artifacts caused by the motion of the subject S is negligible can be selectively obtained. Since such selection is automatically performed, the high-quality vital sign data VD can be easily obtained, and an effect of reducing the burden on the medical worker related to the spot check work can be enhanced.

As another example, the plurality of classes may be set such that the elapsed time from obtaining of the vital sign is associated therewith. For example, it is possible to selectively obtain the vital sign data VD corresponding to a vital sign whose elapsed time from the obtaining is less than a predetermined value, that is, a relatively new vital sign. Since such selection is automatically performed, the vital sign data VD can be easily obtained at a desired timing regardless of the input from the first sensor 111 at any time, and the effect of reducing the burden on the medical worker related to the spot check work can be enhanced.

From the viewpoint of inputting the vital sign at any time, an operation period of the first sensor 111 is preferably longer than a non-operation period. Note that the term “operation period” used here does not necessarily mean a state in which the sensor obtains the vital sign. For example, in a case where the sensor is configured to return from a standby state to start the obtaining of the vital sign under a predetermined condition, a state in which background processing is executed to detect the predetermined condition corresponds to the “operation period” even when an obtaining operation of the vital sign is not performed.

In the case of a visual spot check, it is known that accurate obtaining of a respiratory state is relatively difficult. The vital sign of the subject S obtained by the first sensor 111 preferably includes the respiratory state. In this case, the obtaining of the respiratory state with increased accuracy can be easily automated. Accordingly, the effect of reducing the burden on the medical worker related to the spot check work can be enhanced.

The processor 123 may be configured to obtain an index for evaluating a sign of injury and disease of the subject S, based on the vital sign selected based on the probability output from the inference model 122. Examples of the index include national early warning score (NEWS). NEWS is obtained based on a plurality of types of vital signs. In this case, the vital sign data VD output from the output interface 124 includes information corresponding to the index.

In the case of the spot check through the visit described above, the possibility that a sudden change in the condition of the subject or a sign thereof cannot be grasped cannot be excluded, because the frequency of the spot check is low. By including the index as described above in the vital sign data VD in addition to the input of the vital sign through the first sensor 111 at any time, it is possible to easily grasp a sudden change in the condition of the subject S or a sign thereof at appropriate timing. Accordingly, the effect of reducing the burden on the medical worker related to the spot check work can be enhanced.

As illustrated in FIG. 1, the vital sign obtaining system 10 may include a second sensor 112. The second sensor 112 is configured to output a second detection signal DS2 corresponding to at least one of the level of consciousness or a resting state of the subject S. The second detection signal DS2 may be an analog signal or a digital signal, according to a specification of the second sensor 112. The input interface 121 of the processing device 12 may be configured to receive the second detection signal DS2. The processor 123 may be configured to determine whether to output the vital sign data VD, based on the second detection signal DS2.

As an example, the second sensor 112 may be a camera configured to obtain an image in which the subject S appears. In this case, the second detection signal DS2 may be a signal corresponding to the image. The processor 123 is configured to at least one of the level of consciousness or the resting state of the subject S, using an appropriate image processing technique. For example, the processor 123 may be configured to prohibit the output of the vital sign data VD from the output interface 124, in a case where it is determined that the level of consciousness of the subject S is not suitable for the obtaining of the vital sign, which is, for example, when the subject S is sleeping but needs to be awake. In addition to or instead of this, in a case where it is determined that the subject S is not in the resting state, the processor 123 may be configured to prohibit the output of the vital sign data VD from the output interface 124.

The processor 123 may be configured to determine whether the subject S is appropriately administered with oxygen, based on the image obtained by the second sensor 112.

In another example, the second sensor 112 may be a body motion sensor installed on the hospital bed of the subject S. In this case, the second detection signal DS2 may be a signal corresponding to the body motion of the subject S. The processor 123 is configured to determine the resting state of the subject S, based on the signal. In a case where it is determined that the subject S is not in the resting state, the processor 123 may be configured to prohibit the output of vital sign data VD from output interface 124.

According to such a configuration, it is possible to restrict the output of the vital sign data VD corresponding to a vital sign obtained in an inappropriate situation. Accordingly, the occurrence of a situation in which data based on an inappropriate vital sign is recorded in the management database is suppressed, and a burden of data management related to the spot check work can be reduced.

As illustrated by a broken line in FIG. 1, the second detection signal DS2 output from the second sensor 112 may be input to the management device 13 instead of the processing device 12. In this case, the management device 13 can include an interface and a processor the same as or similar to the input interface 121 and the processor 123 of the processing device 12. The processor of the management device 13 may be configured to determine whether to store the vital sign data VD output from the processing device 12, based on the second detection signal DS2 received by the interface.

For example, in a case where it is determined that the level of consciousness of the subject S is not suitable for obtaining of the vital sign based on the second detection signal DS2, which is, for example, when the subject S is sleeping but needs to be awake, the management device 13 may be configured to prohibit storage of the vital sign data VD received from the processing device 12. In addition to or instead of this, in a case where it is determined that the subject S is not in the resting state, the management device 13 may be configured to prohibit the storage of the vital sign data VD received from the processing device 12.

Even with such a configuration, it is possible to limit the recording of the vital sign data VD corresponding to the vital sign obtained under an inappropriate situation in the management database, and thus the burden of the data management related to the spot check work can be reduced.

The processor 123 of the processing device 12 having various functions described above may be implemented by a general-purpose microprocessor that operates in cooperation with a general-purpose memory. Examples of the general-purpose microprocessor may include a CPU, an MPU, and a GPU. Examples of the general-purpose memory may include a ROM and a RAM.

In this case, the ROM may be configured to store computer programs for implementing the various functions described above. The ROM is an example of a non-transitory computer readable medium that stores a computer program. The general-purpose microprocessor is configured to designate at least part of the programs stored in the ROM, loads the programs into the RAM, and to execute the above-described processing in cooperation with the RAM. The computer program may be pre-installed in the general-purpose memory, or may be downloaded from an external server via a communication network and then installed in the general-purpose memory. In this case, the external server is an example of a non-transitory computer readable medium that stores the computer program.

The processor 123 may be implemented by a dedicated integrated circuit such as a microcontroller, an ASIC, or an FPGA capable of executing the computer program. In this case, the computer program is pre-installed in a storage element included in the dedicated integrated circuit. The storage element is an example of a computer-readable medium that stores the computer program. The processor 123 may also be implemented by a combination of a general-purpose microprocessor and a dedicated integrated circuit.

The various configurations described above are merely examples for facilitating understanding of the present disclosure. Each configuration example can be appropriately changed or combined within the scope of the gist of the present disclosure.

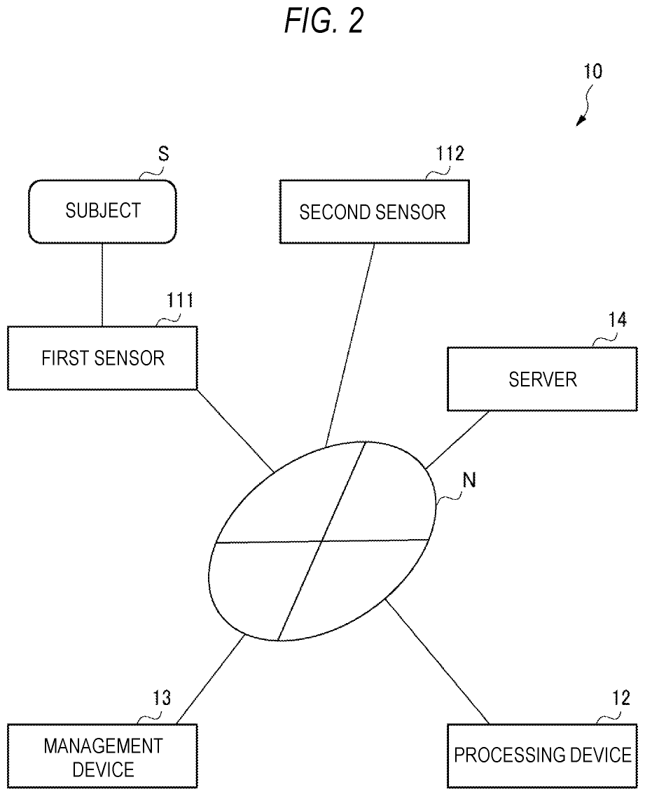

The inference model 122 is not necessarily mounted on the processing device 12. As illustrated in FIG. 2, the vital sign obtaining system 10 may have a configuration in which the first sensor 111, the second sensor 112, the processing device 12, the management device 13, and the server 14 are communicably connected via a communication network N. In this case, if data can be exchanged with the processor 123 of the processing device 12, the inference model 122 can be mounted on the management device 13 or the server 14.

The configurations listed below also constitute a part of the present disclosure.

(1) A vital sign obtaining device including:
an interface configured to receive, from a first sensor, a first detection signal corresponding to a vital sign of a subject, the first sensor configured to obtain the vital sign;
an inference model configured to output a probability that the first detection signal is classified into each of a plurality of classes; and
a processor configured to output data corresponding to a vital sign associated with the first detection signal, the probability that the first detection signal is classified into one of the plurality of classes being equal to or greater than a threshold.

(2) The vital sign obtaining device according to the above described (1),
in which one of the plurality of classes is associated with an artifact superimposed on the vital sign.

(3) The vital sign obtaining device according to the above described (1) or (2),
in which one of the plurality of classes is associated with an elapsed time from obtaining of the vital sign.

(4) The vital sign obtaining device according to any one of the above described (1) to (3),
in which the vital sign includes respiration information.

(5) The vital sign obtaining device according to any one of the above described (1) to (4),
in which the data includes an index for evaluating a disease sign of the subject.

(6) The vital sign obtaining device according to any one of the above described (1) to (5),
in which the interface is configured to receive, from a second sensor, a second detection signal corresponding to at least one of a level of consciousness of the subject or a resting state of the subject, and
in which the processor is configured to determine whether to output the data, based on the second detection signal.

(7) A non-transitory computer readable storage medium storing a computer program. the computer program including instructions which, when executed by a processor mounted on a vital sign obtaining device, cause the vital sign obtaining device to:
receive, from a sensor, a detection signal corresponding to a vital sign of a subject, the sensor configured to obtain the vital sign;
input the detection signal to an inference model, the inference model being configured to output a probability that the detection signal is classified into each of a plurality of classes; and
output data corresponding to a vital sign associated with the detection signal, the probability that the detection signal is classified into one of the plurality of classes being equal to or greater than a threshold, the probability being output from the inference model.

(8) A vital sign obtaining system including:
a first sensor configured to output a first detection signal corresponding to a vital sign of a subject;
a management device configured to manage attribute information of the subject;
an inference model configured to output a probability that the first detection signal is classified into each of a plurality of classes; and
a processor configured to output, to the management device, data corresponding to a vital sign associated with the first detection signal, the probability that the first detection signal is classified into one of the plurality of classes being equal to or greater than a threshold,
in which the management device is configured to store the data in association with the attribute information.

(9) The vital sign obtaining system according to the above described (8),
in which the first sensor has an operation period longer than a non-operation period.

(10) The vital sign obtaining system according to the above described (8) or (9), further including:
a second sensor configured to output a second detection signal corresponding to at least one of a level of consciousness of the subject or a resting state of the subject,
in which the processing device is configured to determine whether to output the data to the management device, based on the second detection signal.

(11) The vital sign obtaining system according to the above described (8) or (9), further including:
a second sensor configured to output a second detection signal corresponding to at least one of a level of consciousness of the subject or a resting state of the subject,
in which the management device is configured to determine whether to store the data, based on the second detection signal.

(12) The vital sign obtaining system according to any one of the above described (8) to (11)
in which the management device is configured to give, to the data, an index for evaluating a disease sign of the subject.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A vital sign obtaining device comprising:
an interface configured to receive, from a first sensor, a first detection signal corresponding to a vital sign of a subject, the first sensor configured to obtain the vital sign;
an inference model configured to output a probability that the first detection signal is classified into each of a plurality of classes; and
a processor configured to output data corresponding to a vital sign associated with the first detection signal, the probability that the first detection signal is classified into one of the plurality of classes being equal to or greater than a threshold.

2. The vital sign obtaining device according to claim 1,
wherein one of the plurality of classes is associated with an artifact superimposed on the vital sign.

3. The vital sign obtaining device according to claim 1,
wherein one of the plurality of classes is associated with an elapsed time from obtaining of the vital sign.

4. The vital sign obtaining device according to claim 1,
wherein the vital sign includes respiration information.

5. The vital sign obtaining device according to claim 1,
wherein the data includes an index for evaluating a disease sign of the subject.

6. The vital sign obtaining device according to claim 1,
wherein the interface is configured to receive, from a second sensor, a second detection signal corresponding to at least one of a level of consciousness of the subject or a resting state of the subject, and
wherein the processor is configured to determine whether to output the data, based on the second detection signal.

7. A non-transitory computer readable storage medium storing a computer program, the computer program comprising instructions which, when executed by a processor mounted on a vital sign obtaining device, cause the vital sign obtaining device to:
receive, from a sensor, a detection signal corresponding to a vital sign of a subject, the sensor configured to obtain the vital sign;
input the detection signal to an inference model, the inference model being configured to output a probability that the detection signal is classified into each of a plurality of classes; and
output data corresponding to a vital sign associated with the detection signal, the probability that the detection signal is classified into one of the plurality of classes being equal to or greater than a threshold, the probability being output from the inference model.

8. A vital sign obtaining system comprising:
a first sensor configured to output a first detection signal corresponding to a vital sign of a subject;
a management device configured to manage attribute information of the subject;
an inference model configured to output a probability that the first detection signal is classified into each of a plurality of classes; and
a processor configured to output, to the management device, data corresponding to a vital sign associated with the first detection signal, the probability that the first detection signal is classified into one of the plurality of classes being equal to or greater than a threshold,
wherein the management device is configured to store the data in association with the attribute information.

9. The vital sign obtaining system according to claim 8,
wherein the first sensor has an operation period longer than a non-operation period.

10. The vital sign obtaining system according to claim 8, further comprising:
a second sensor configured to output a second detection signal corresponding to at least one of a level of consciousness of the subject or a resting state of the subject,
wherein the processing device is configured to determine whether to output the data to the management device, based on the second detection signal.

11. The vital sign obtaining system according to claim 8, further comprising:
a second sensor configured to output a second detection signal corresponding to at least one of a level of consciousness of the subject or a resting state of the subject,
wherein the management device is configured to determine whether to store the data, based on the second detection signal.

12. The vital sign obtaining system according to claim 8,
wherein the management device is configured to give, to the data, an index for evaluating a disease sign of the subject.
